(19) United States
(12) Patent Application Publication
(10) Pub. No.: US 2014/0128755 A1
(43) Pub. Date: May 8, 2014

(54) Pulse Detecting Device and Method

(71) Applicants: Quinn Snyder, Phoenix, AZ (US); Nolan R. Morris, Portland, OR (US); Douglas A. Morris, Portland, OR (US)

(72) Inventors: Quinn Snyder, Phoenix, AZ (US); Nolan R. Morris, Portland, OR (US); Douglas A. Morris, Portland, OR (US)

(21) Appl. No.: 13/672,504

(22) Filed: Nov. 8, 2012

(51) Int. Cl. A61B 5/024

(52) U.S. Cl. CPC A61B 5/02444
USPC 600/502

(57) ABSTRACT

The present invention includes a system and method for detecting a pulse. The system includes a sound sensor unit device having a self-adhesive material and a sensor body with a sound chamber, a power source, a MEMS microphone and a transmitting device. The sensor unit wirelessly sends a first signal to a remote unit adapted to receive the first signal. The remote unit includes a housing having a receiver, an audio/visual interface unit whereby the audio visual interface unit processes the first signal and translates the first signal into output formats to an audible signal and to a visual display signal, and a power source in electrical communication with the receiver and the audio visual interface unit

### BACKGROUND

The present invention relates to electromechanical sensing devices for interfacing with the human body and, more specifically, the present invention, in various preferred embodiments relates to a sound-based and audible pulse detection system, method, and device.

The need for technical and product advances to assist medical personnel and first responders in the early stages of a crisis cannot be overstated. During the initial assessment of a patient, one of the urgent and primary needs is to verify that the patient has a pulse. Although the manual technique for finding a pulse commonly used today is relatively simple and well established by medical professionals and first responders, it is often difficult and frustrating to master due to environmental, situational, and uncontrolled variables. Noisy conditions, clothing, or other physical properties of the patent along with variable environmental settings can make this seemingly simple task difficult and stressful. As a result, there is a need for a simple device that can quickly and easily be used to verify that a pulse exists on a patient in the field so that first responders and medical personnel can properly proceed with appropriate emergency medical procedures.

Since the stethoscope, medical science has attempted to improve methods and devices to detect a patient's pulse. Such attempts try to increase accuracy and repeatability. But, sound detection of pulse remains a preferred method, and so the art is replete with examples of enhancing sound detection. For example, U.S. Pat. No. 2,831,479 to Briskier and issued on 1958-04-22 (the entire contents of which are hereby incorporated by reference and included as if fully set out herein) describes the use of a microphone placed on a patient's chest and the microphone being coupled to a rotary counter for pulse rate display. Further, the utilization of analog signals corresponding to the heartbeat of a subject is also well documented in U.S. Pat. No. 3,908,636 to Page issued on 1975-09-30 (the entire contents of which are hereby incorporated by reference and included as if fully set out herein). Page describes a device that touches the outside of a patient's finger and amplifies the sound signal and outputs that signal to a speaker.

Yet another attempt to detect pulse at a patient's finger and amplify that sound is described by Hardy et al. in U.S. Pat. No. 4,038,976 issued on 1977-08-02 and includes a visible flash on a device worn on the wrist, where the wrist device is wired to the sensing device on the finger.

Wireless communication of physiological data by means of a sensor to a receiver via a transmitter is generally understood in this art. For example, U.S. Pat. No. 3,949,388 to Fuller on 1976-04-06 describes a narrow frequency (RF) spectrum transmitter adapted to transmit data from a thermistor adhered to a patient.

Remote display of sensed patient data is also generally known in this art. For example, U.S. Pat. No. 5,387,194 to Williams et al. issued 1995-02-07 describes a pressure transducer positioned pressure in a syringe detecting pressure difference in a catheter, the pressure difference is transmitted by infrared light pulses to a remote monitor in sight distance of the transmitter.

Despite advances in sound-detecting pulse methods and devices, there remains a need for a device that is adapted for use in the urgent and early diagnosis of a patient—when it is vital to quickly detect and verify the presence of a pulse. Such a device should be versatile for various emergency and non-emergency uses including use in hospital emergency rooms, ambulances, hospitals, and by first responders wherever accidents may occur. There is a need for a device to standardize care for emergency first responders and should be easy to use, reliable, and to have the flexibility for enhanced product capabilities such as data storage, location and time record stamping, and information transfer to computer storage devices to insure that each event or session can be fully documented. The device should also have a flexible architecture for future enhancements and improvements; for example, allowing for engaging multiple sensors or microphones, of various technologies, to enhance the sensitivity and pick up of the heart beat as well as the ability to monitor and track heart rate changes and to include adaptations for real-time wireless data transfer, GPS/time/date stamping, and use of the sensor component with other hospital equipment, for example.

### SUMMARY OF THE INVENTION

In one contemplated embodiment, the present invention includes a pulse detecting device comprising an integrated circuit comprising a processor coupled to memory, at least one data input port coupled to the processor and at least on output port coupled to the processor; a means for sensing sound comprising a sound sensor coupled to the integrated circuit; a means for processing the sound sensed by the sound sensor, the means comprising an audio processing circuitry and processor-implemented algorithm adapted to interface with the integrated circuit; a means for audio and/or visual interface coupled to the at least one output port, the means comprising any combination of a speaker, a display, or both; and a power source coupled to the integrated circuit and adapted to distribute power as required by each means.

The pulse-detecting device (or sensor device) includes the means for sensing sound, which includes a diaphragm covering a sound chamber and an omni-directional microphone comprising a solid state MEMS device that picks up the sound on a silicon chip and then converts the sound to electrical signals adapted to be received by the processor.

One suitable integrated circuit further comprises a TI TMS 32005515 Digital Signal Processor chip.

One contemplated method using a pulse detecting system includes providing a device having a sensor unit and a remote unit wherein the sensor unit and remote unit are in wireless communication and are physically separate from each other. Then, place the sensor unit a subject patient to detect sound indicating a pulse.

A device for detecting pulse in a patient includes an integrated circuit comprising a processor coupled to memory, at least one data input port coupled to the processor and at least on output port coupled to the processor, a sound sensor coupled to the integrated circuit, audio processing circuitry and processor-implemented algorithm adapted to interface with the integrated circuit, a speaker, a display, or both and a power source coupled to the integrated circuit and adapted to distribute power as required. The sound sensor includes a diaphragm covering a sound chamber; and an omni-directional microphone comprising a solid state MEMS device that picks up the sound on a silicon chip and then converts the sound to electrical signals adapted to be received by the processor.

### BRIEF DESCRIPTION OF THE DRAWING

FIG. 1 is a block diagram of a system according to the present invention.

FIG. 2 is a representation of a system according to one embodiment of the present invention.

FIG. 3 is block diagram of a remote device according to one embodiment of the present invention.

FIG. 4 is an illustration of one preferred method step using a device according to the present invention.

FIG. 5 is an illustration of another preferred method step using a device according to the present invention

FIG. 6 is a representative touch-screen view according to one embodiment of the present invention.

FIG. 7 is a block diagram of a sensor unit according to one embodiment of the present invention.

### DESCRIPTION OF THE INVENTION

Possible embodiments will now be described with reference to the drawings and those skilled in the art will understand that alternative configurations and combinations of components may be substituted without subtracting from the invention. Also, in some figures certain components are omitted to more clearly illustrate the invention.

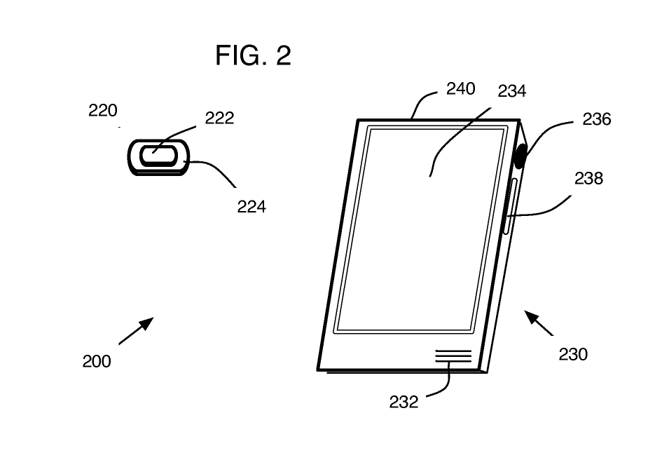

The present invention, in various preferred embodiments relates to a sound-based pulse detection system 200, method, and device. FIG. 2 illustrates one such contemplated system. In one embodiment, a system 200 includes a sensor device 220 adapted for use on human patients, and more specifically, the sensor device is adapted for use externally, temporarily securing to the skin over and in close proximity to a major artery, such as the carotid artery. The sensor device utilizes the sound of the patient's pulse as detected over the artery and then sends an electronic signal, wirelessly, to a remote device 230 where the signal is converted into both audible sound through playback speakers 232 and a visual pulse display viewable on an output screen 234. Contemplated embodiments of this remote device include utilizing existing hand-held computer devices such as the iPad (a registered trademark of Apple, Inc. of San Jose, Calif., USA), iPod, iPhone, other tablet computers, smart phones, and related components that include a screen display and audio output (headphone jack 236 and/or internal speaker 232), and include a wireless communication means such as Bluetooth, WIFI, or other similar wireless communication systems as would be well-understood by those having ordinary skill in this art.

FIG. 2 further shows this preferred embodiment, which includes the sensor device 220, which is a small, wearable device, and is termed an Emergency Pulse Detector (EPD) that can detect a pulse and relay a signal of the pulse, and to amplify the signal and translate the pulse date to both visual and audible outputs. This embodiment includes four main components: a sound sensor, an audio processing unit, an audio/visual interface unit, and a power source.

In this first preferred embodiment, the sound sensor device 220 is adapted for use on a patient, specifically to be placed over the artery or vein where the pulse is to be detected. The sound sensor includes a housing 222 having a diaphragm covering a sound chamber. The sound chamber amplifies sound, which, in turn, is detected by a microphone consisting of a diaphragm covered MEM's chip. This contemplated microphone is a small solid state MEMS device that picks up the sound on a silicon chip and then converts the sound to electrical signals.

The sound chamber acts like a traditional stethoscope bell in that it collects sound, as would be familiar in a traditional stethoscope. However, the sound chamber enables the sensor device to convert the sound and distribute it digitally and wirelessly, rather than mechanically directing sound waves through the rubber branches of a stethoscope. In a first embodiment, pulse is detected using a stethoscope microphone integrated into the sensor assembly 220. A number of sensor technologies may be incorporated into the sensor including a wide-range of electromechanical sensors, which allow for the pulse signal to be directed and then process, and adapting such sensors are well-understood to those skilled in this art.

The electrical signals are processed, which includes noise reduction and signal amplification, then transmitted or sent to the audio processing unit. In this contemplated embodiment, the audio processing unit is located separate from and remotely with respect to the sound sensor and, as such, it includes its own housing, or base unit housing. This base unit housing encapsulates the audio processing unit, a power supply, and various signal out put means including at least one loudspeaker, a sound output jack, a display screen, and a port for interfacing the data contained on the unit to other peripheral devices. This port can be a physical port, such as a USB port, or could be a wireless transmitter, such as WIFI, RF, light, or Bluetooth, for example.

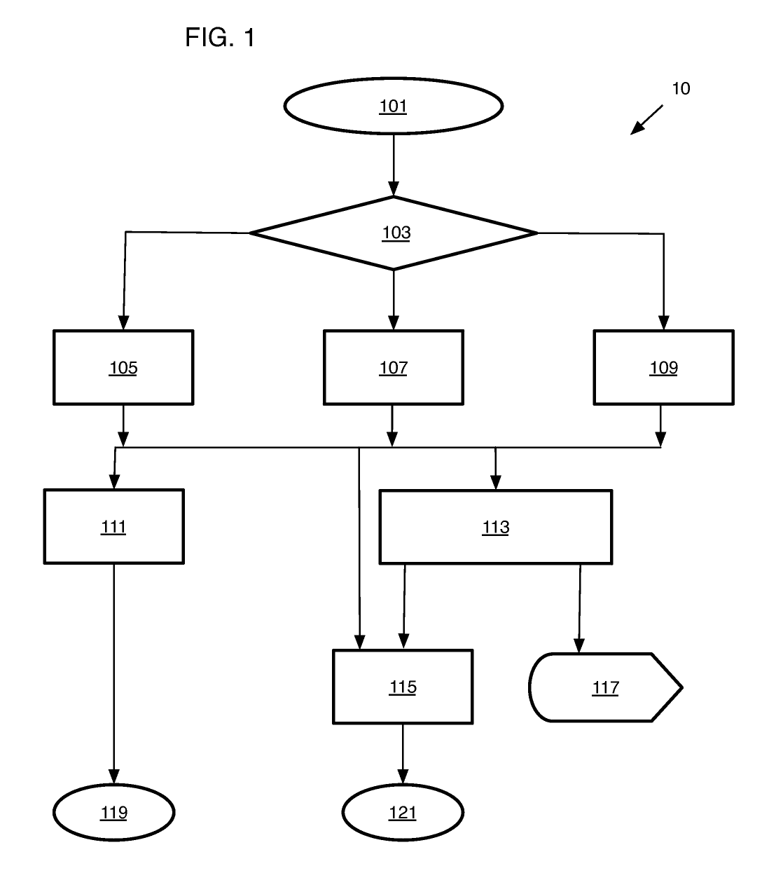

In this first embodiment, the audio processing unit, as FIG. 1 shows, includes audio processing circuitry 10, of which many commercially available sound sensors 101 are well suited to this use in the present invention. Without limiting the scope and intent of the present invention, some contemplated sound sensors include infrared, electromechanical, piezoelectric, bio-potential, laser, and others, for example. However, it will be appreciated by those skilled in the art that other sensor technologies would work equally well. Such technologies include, but are not limited to, Piezo film sensors, optical sensors, microphones, infrared sensors, sound sensors, and amplifiers.

Referring specifically to FIG. 1, the audio processing unit 10 further includes a receiver for receiving the electrical or data signals from the sound sensor and these signals are transmitted from the CODEC (part of the Bluetooth chipset), which is represented by block 101 in FIG. 1. Based on a user-selected mode (Block 103) this input is filtered and conditioned. The contemplated filter and conditioning of signals is represented by block 105 for FIR filter for Bell Mode, Block 107 for FIR filter for Diaphragm Mode, or Block 109 FIR filter for extended mode, these modes are defined in greater detail, below. The filtering, conditioning, and or amplifying of those signals could occur on the sensor unit or on the audio processing unit, or divided between both. In this first preferred embodiment, however, the audio processing circuit is adapted to receive the signals from the sound sensor and then filters and conditions the signal based on algorithms utilized in a digital signal processing microcontroller. User-interface switches (Block 103) allow for the change of audible pick up frequencies depending on need for sensitivity from 20 Hz up to 6000 Hz, for example. The microcontroller/chip set allows the overall design to operate on low power to preserve battery life.

The sensor unit further includes a volume control (Block 111) that amplifies the sound signal after the filtering and transmits that amplified sound signal to CODEC 119 for subsequent use, as further described herein. The sensor unit further manipulates the signal after the filtering of any one of blocks 105 107 or 109 and translates (Block 115 a UART Transmission module) the sound wave into signals to be processed by a visual display unit (Block 121), such as a PC display so the wave can be visualized as a line graph, for example. This post filtering signal (line 106) is also fed to a heart-rate calculation module or subroutine 113, which in turn is fed to the PC display 121 or to a LCD display 117, for example on the remote unit.

Once the audio processing circuit has performed the filtering and conditioning of the electrical signal from the sound sensor, this new signal is transmitted to an audio/visual interface. In this first preferred embodiment, this new signal is used directly to electrically power the audio/visual interface device. However, it is also contemplated that this new signal can be transmitted by light, wireless, or wired communication means to an audio/visual interface device that uses its own source of power and own receiver and own logic to translate this new signal to a format suitable for output on the device's own output means such as a screen, on-board loud speaker, speaker jack, or visual output jack, for example.

In this first preferred embodiment the remote or base unit also includes the Audio/Visual interface, which in turn includes at least one output speaker and a display, such as a color LCD display, for example. The at least one speaker produces amplified sound equivalent to the pulse being detected. Volume control is achieved with user interface switches, which can be mechanical switches or soft-switches on a touch screen, for example.

In this first preferred embodiment, the display consists of a color LCD touch-screen display, which is configurable by pre-programmed subroutines and is alterable based on input received either from the user of the device, from patient condition, or other selectable parameter. The display displays a variety of information including the pulse rate in a beats per minute.

Data can be stored on the audio processing unit, and this data can be downloaded, either real-time, or at a later time, to other devices. Accordingly, additional viewing of the actual pulse signal can be achieved by either physically connecting the audio processing unit by a cable to a peripheral device or by transmitting a wireless signal to a peripheral device. In this first preferred embodiment, the audio processing unit couples by a cable to a computer's COM port. Software on board the audio processing unit, and or software on the computer, or a combination of both, will take and translate data from the audio processing unit to enable subsequent processing and display of that data in user-friendly modes, such as audible output, or visual output including color-coded and time-sequenced pulses, line graphs, and other techniques as would be well-understood in this art.

One contemplated visual output includes a display of the monitored heart beat. Other outputs, which can be hosted on the audio processing unit or on another peripheral device can be quite complex, incorporating LCD display information on pulse rates, timed sessions, data and date/time stamping, and other parameters of the session.

Further, this contemplated invention includes multiple channeling in the audio processing unit, which enables simultaneous use of multiple sound sensing units. Accordingly, this preferred embodiment contemplates three channel inputs for multiple sensors, audio output in three selectable modes including a Bell mode (20 hz to 220 hz), a Diaphragm mode (50 hz to 600 hz), and an Extended range mode (20 hz to 2000 hz).

The housing of the remote or base unit also includes volume controls and external speaker output jack including sound amplification, real time display of heart signal, store and playback of signals internally or for storage on PC or computer systems via USB connection options, wireless data transmission and storage, GPS positioning and time stamping, and multiple display technologies for reduced current drain.

The sound sensor includes an on-board power source. The base unit containing the audio processing unit contains a separate power source. Suitable power sources for either of these units include AA or AAA batteries, rechargeable lithium Ion battery pack with charger, primary coin cells for small size and long battery life, or hybrid batteries that combine rechargeable batteries with solar, piezo electric, or thermoelectric generators to supplement the battery storage, for example.

In one contemplated embodiment, the device includes a flexible design architecture, which is accomplished by using the TI TMS 32005515 Digital Signal Processor chip, allows for engaging multiple sensors or microphones, of various technologies, to enhance the sensitivity and pick up of the heart beat as well as the ability to monitor and track heart rate changes. These and other features will be incorporated in a family of products that share the spirit and scope the invention as contemplated herein.

The device has been designed to accommodate a number of different data storage techniques to insure that a record of any patient event can be captured and documented easily. Information will include a log of the pulse readings over the entire period of time the data is acquired for each patient. The data will include the pulse rate via actual audio recordings (pulses as they occur over time) along with a time and date stamp and GPS location. Patient information can be added manually or via bar code. Data can be stored locally on the EPD and then transferred to more permanent storage systems via USB connection or wirelessly via Bluetooth or other RF protocols.

FIG. 2 illustrates the pulse detecting system 200, which includes a sensor device 220 consisting of a housing 222 having a sound chamber and processing circuitry (of FIG. 1, for example). And the housing attaches or couples to an adhesive pad 224 adapted for the selective placement of the sensor unit over an artery on the patient. The remote device 230 has an exterior housing 240 that includes an output screen 234, an output speaker 232, a headphone jack 236 and memory port, such as a USB slot 238. Internally and not shown in this Figure, the housing 240 includes a power supply, control logic circuitry, memory, wireless communication means, and signal connectivity means between all the components, as would be well-understood by those of ordinary skill in the art.

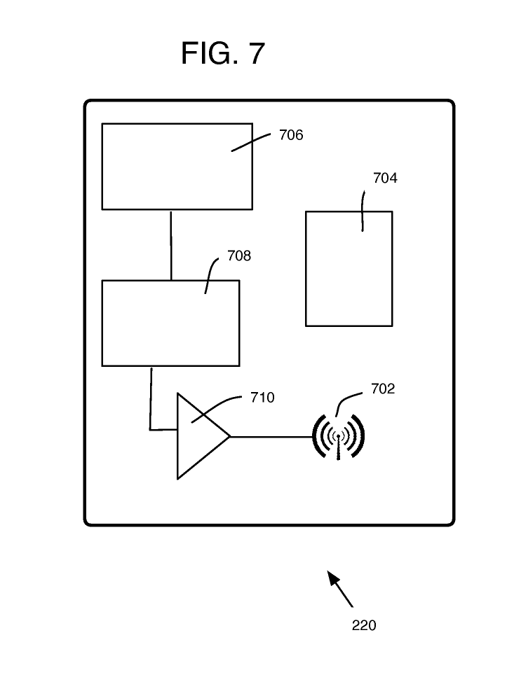

FIG. 7 illustrates components of the sensor device 220, which includes the means for sensing sound, which includes a diaphragm covering a sound chamber 706 and an omni-directional microphone 708 comprising a solid state MEMS 710 device that picks up the sound on a silicon chip and then converts the sound to electrical signals adapted to be received by the processor. The sensor includes a power source 704 and wireless communication means, such as a Bluetooth antenna 702, for example.

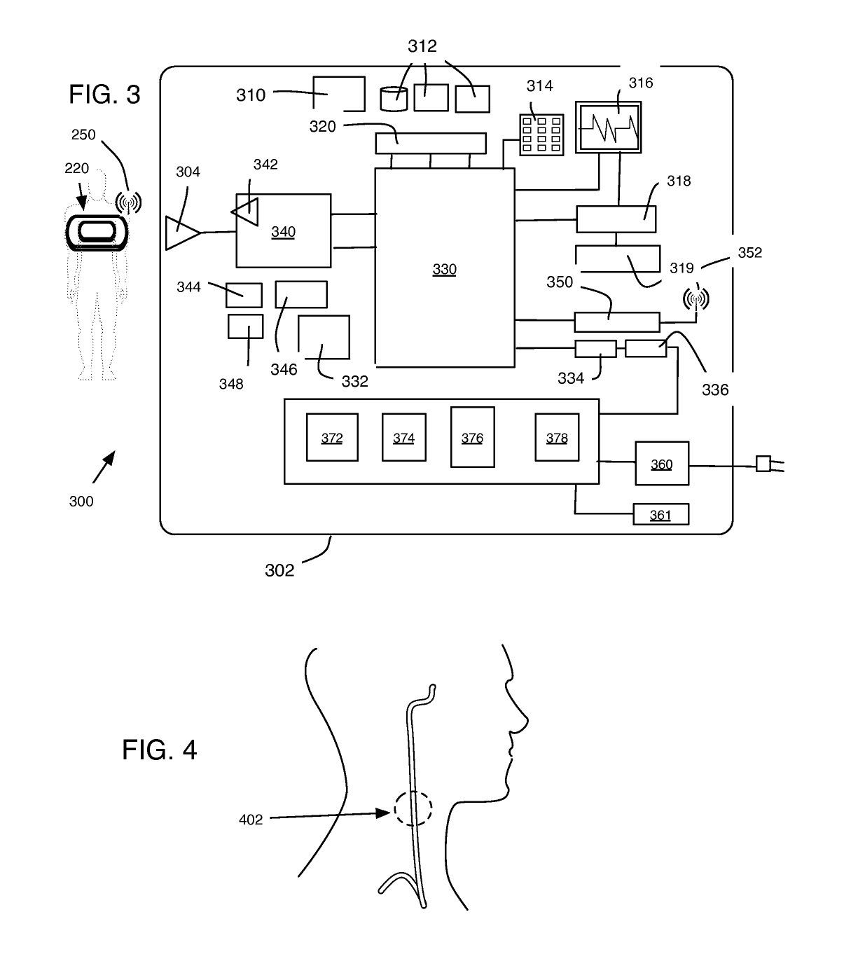

FIG. 3 is a schematic diagram illustrating the internal components of the remote device relative to the sensor device 220. The sensor device includes mean for wireless transmission 250 such as a blue tooth antenna and processing circuit and power source for transmitting. The sensor device first captures sound relating to a pulse when the sensor device is placed over an artery. The sound wave produced by the pulse is directed into an internal sound chamber, as previously discussed, and translates that into a signal (See FIG. 1). This signal is than wirelessly transmitted to the remote device 302. The remote device is adapted to receive the signal from the sensor device, manipulate that signal so it can be output in various ways. The remote device includes a processor 330 for controlling all the functions necessary to receive the sensor signal and translate that signal into various components. A wireless communication means 350, such as a blue tooth antenna adapted to send and receive signals is in signal communication with the processor 330. Additionally, the processor 330 accesses ROM member 320 and RAM memory 312 and such memory includes dedicated power 310, as would be understood in the art.

The remote device 302 also includes data input and output capability. Conventional keypad 314 or a touch screen 318 with backlight 319 enable a user to input instructions, select subroutines, invoke applications, and otherwise manipulate data into the remote device and can be used to configure both the remote device and the sensor device.

The processor, also in signal communication with a clock 346, REF 344, and logic controller 348.

The remote device 302 includes on-board power (system power) 378 such as a battery 360, and can be charged from an external source, or use external source power from an AC/DC adapter 361. The remote device 302 contemplates a battery management system 370 consisting of a lithium battery 372, charger 374, and battery level 376 assessment logic to manage charging (to avoid overcharging and to avoid early charging) of the battery 372.

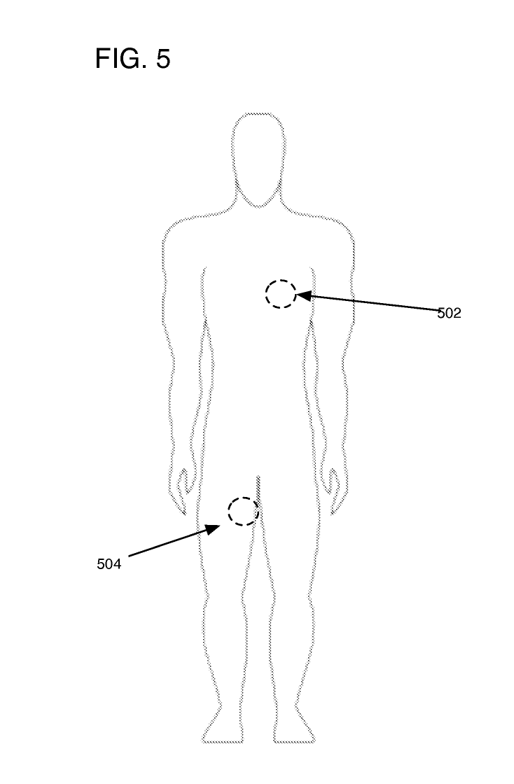

FIGS. 4 and 5 illustrated contemplated points for placing the sensor device to capture an accurate pulse rate. Such placement points include, but are not limited to, the carotid artery 403, the inner groin (femoral artery) 504 and over the chest 502, over a 3.6″ radius area.

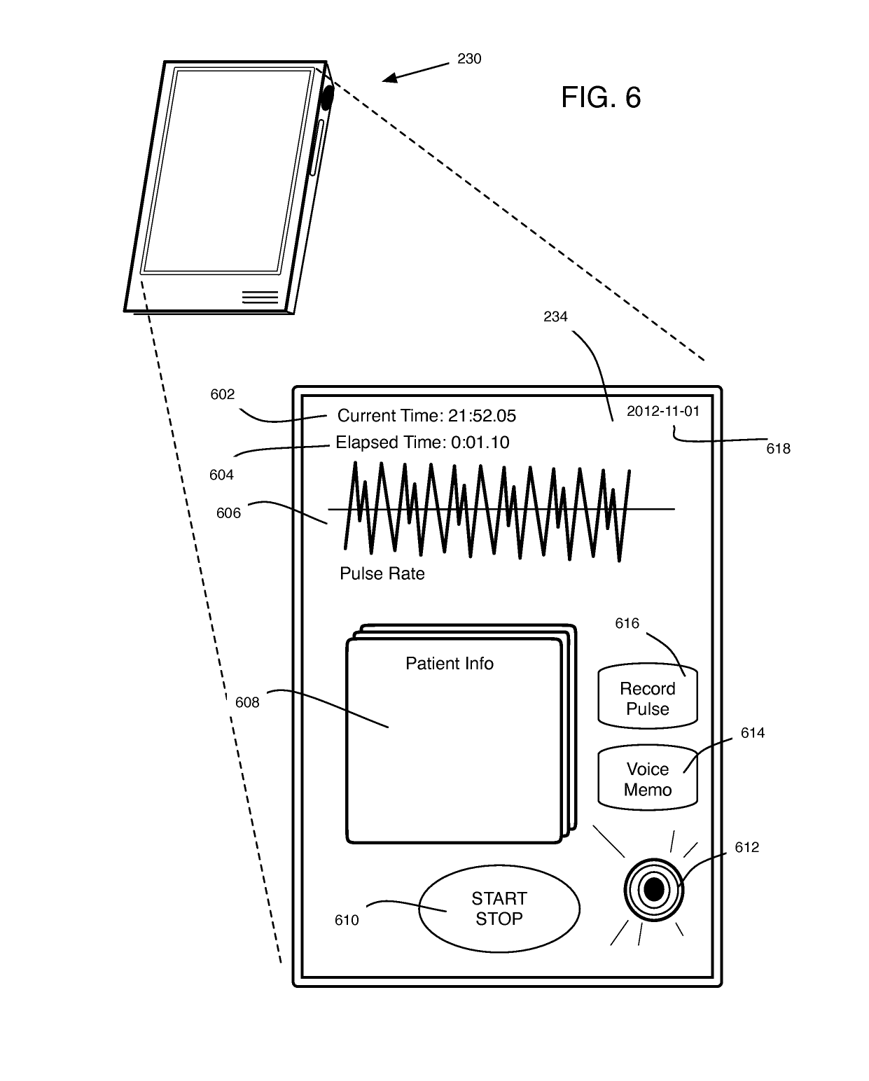

FIG. 6 illustrates a possible touch-screen input output (screen shot) on a remote device 200 using the teachings of the present invention. The touch-screen 234 can display, based on pulse data collected and transmitted by the sensor device (not shown in this figure) and manipulated the software, hardware, and firmware on the remote device, a graph 606 of the pulse, a indicator light that pulses with the pulse rate 612, the current time 602, the elapsed time 604, the current date 618. Further, the screen 234 configures to enable touch-screen applications, such as patient information 608, that can be used to input particular information on a given patient and/or display useful information about the patient being treated, for example. The touch screen also configures to have virtual buttons that can start recording streaming data of the hear rate, start or stop the monitoring of the patient's heart rate, begin recording voice data from an integrated microphone, and other functions, for example.

Further, although the preferred and contemplated embodiments of the present invention as disclosed herein enumerate particular components currently commercially available, it will be appreciated by those of skill in this art that substitutions of make, brand, and the like can be made without detracting from the spirit and scope of the present invention.

Although the invention has been particularly shown and described with reference to certain embodiments, it will be understood by those skilled in the art that various changes in form and detail may be made without departing from the spirit and scope of the invention. And, although claims are not required, we claim at least:

We claim:

1. A system for detecting a pulse, the system comprising:
a sound sensor adapted to convert audible or sound-based pulse to a digital, first signal; and
a remote unit in wireless communication with the sound sensor adapted to receive the digital, first signal.

2. The system of claim 1 wherein:
the sound sensor comprises a sound detection circuit comprising a microphone in signal communication with a noise-ratio management means and a signal amplification means and a wireless transmission means wherein the first, digital signal, prior to transmitting the digital, first signal is processed by any combination of noise-ratio management and signal amplification prior to being transmitted to the remote unit.

3. The system of claim 2 wherein:
the wireless transmission means comprises a Bluetooth transmitting circuit.

4. The system of claim 1 wherein:
the remote unit comprises a data storage memory and export communication means in signal communication with the sound sensor whereby the digital, first signal can be stored and selectively downloaded by the export communication means on demand.

5. The system of claim 1 wherein:
the sound sensor comprises a sound chamber in sound communication with the microphone.

6. The system of claim 1 wherein wireless communication between the sound sensor and the remote unit comprises:
a Bluetooth sending circuit in signal communication with the microphone on the sound sensor.

7. A system for detecting a pulse, the system comprising:
a sound sensor unit device comprising an adhesive pad comprising a first surface having a self-adhesive material and a sensor body adapted to selectively couple to the adhesive pad, the sensor body adapted to contain sound chamber, a power source, a MEMS microphone and a transmitting device, and wherein the sound chamber is arranged adjacent to a bottom wall of the sensor body and adapted to transfer at least one sound wave to the MEMS microphone, the MEMS microphone being adapted to translate the at least one sound wave from the sound chamber to a first signal and the MEMs microphone being in signal communication with the transmitting device enables the transmitting device to transmit the first signal; and
a remote unit adapted to receive the first signal.

8. The system of claim 6 wherein the remote unit further comprises:
a remote unit housing adapted to contain a receiver, the receiver adapted to receive the first signal, the receiver in communication with an audio/visual interface unit whereby the audio visual interface unit processes the first signal and translates the first signal into output formats to an audible signal and to a visual display signal;
and a power source in electrical communication with the receiver and the audio visual interface unit.

9. The system of claim 7 wherein the remote unit further comprises:
at least one audio output means contained in the remote unit housing, the at least one audio output means comprising any combination of at least one loudspeaker, an audio output jack, or a wireless communication means wherein the wireless communication means comprises any combination of a WIFI transmitter, an IR light transmitter, an RF transmitter or a Bluetooth transmitter; and
at least one visual display out means contained in the remote unit housing, the visual display means comprising a LCD screen.

10. The system of claim 7 wherein:
the audio visual interface unit further comprises a memory device for storing data associated with any combination of the first signal, output formats for an audible signal, or output formats for a visual display signal; and
a means for outputting the data to a peripheral device.

11. A method for detecting pulse comprising:
providing a device for detecting comprising
a sound sensor unit device comprising a sensor body, the sensor body adapted to contain sound chamber, a power source, a MEMS microphone, and a wireless transmitting device, wherein the MEMS microphone being adapted to translate the at least one sound wave from the sound chamber to a first signal and the MEMs microphone being in signal communication with the transmitting device enables the transmitting device to transmit the first signal; and a remote unit adapted to receive the first signal.
placing the sound sensor unit device near a pulse;
transmitting a first signal from the sound sensor unit device to the remote unit device; and
translating the first signal in the remote device and outputting at least one second signal wherein the at least one second signal include any one of the following, an audible output, a visual output in the form of a graphical representation of pulse over time, or a visual output of a flashing light effect.
